(19) United States
(12) Patent Application Publication
(10) Pub. No.: US 2005/0233378 A1
(43) Pub. Date: Oct. 20, 2005

(54) Biosensor

(75) Inventor: Toshiaki Kubo, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/086,582

(22) Filed: Mar. 23, 2005

(30) Foreign Application Priority Data
Mar. 26, 2004 (JP) 2004-093046
Mar. 26, 2004 (JP) 2004-093047

(51) Int. Cl. G01N 33/53; C12M 1/34

(52) U.S. Cl. 435/7.1; 435/86; 435/287.2

(57) ABSTRACT

It is an object of the present invention to provide a detection surface used for biosensors, which has an improved efficiency of immobilizing a physiologically active substance. The present invention provides a biosensor having a substrate, to the surface of which a compound represented by the following formula (A) is bound: 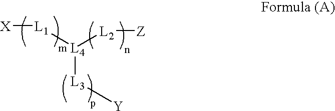
wherein X represents a group capable of binding to the surface of a biosensor or a functional group existing on the surface of the biosensor; Y represents a group capable of binding to a physiologically active substance or a compound binding to the physiologically active substance; Z represents a group having an electric charge; each of L₁, L₂, and L₃ independently represents a divalent linking group; L₄ represents a trivalent group; and each of m, n, and p independently represents an integer of 0 or greater.

### TECHNICAL FIELD

The present invention relates to a biosensor and a method for analyzing an interaction between biomolecules using the biosensor. Particularly, the present invention relates to a biosensor which is used for a surface plasmon resonance biosensor and a method for analyzing an interaction between biomolecules using the biosensor.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The OCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

As a thin film having a functional group capable of immobilizing a physiologically active substance, there has been reported a measurement chip where a physiologically active substance is immobilized by using a functional group binding to metal, a linker with a chain length of 10 or more atoms, and a compound having a functional group capable of binding to the physiologically active substance (Japanese Patent No. 2815120). Moreover, a measurement chip comprising a metal film and a plasma-polymerized film formed on the metal film has been reported (Japanese Patent Laid-Open No. 9-264843).

The immobilization efficiency of the aforementioned measurement chip significantly decreases depending on the isoelectric point of a physiologically active substance. In such a case, immobilization is carried out in a buffer solution having a lower pH value than the isoelectric point. However, since some physiologically active substances become unstable due to change in the pH value, an anchoring group having a good immobilization efficiency has been desired.

### DISCLOSURE OF THE INVENTION

It is an object of the present invention to solve the problems of the prior art techniques. In other words, it is an object of the present invention to provide a detection surface used for biosensors, which has an improved efficiency of immobilizing a physiologically active substance.

As a result of intensive studies directed towards achieving the aforementioned object, the present inventors have found that a detection surface used for biosensors having an improved efficiency of immobilizing a physiologically active substance can be obtained by introducing a group having an electric charge into a substrate, thereby completing the present invention.

Thus, the present invention provides a biosensor having a substrate, to the surface of which a compound represented by the following formula (A) is bound: 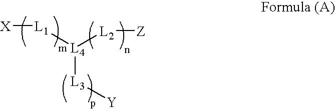
wherein X represents a group capable of binding to the surface of a biosensor or a functional group existing on the surface of the biosensor; Y represents a group capable of binding to a physiologically active substance or a compound binding to the physiologically active substance; Z represents a group having an electric charge; each of L₁, L₂, and L₃ independently represents a divalent linking group; L₄ represents a trivalent group; and each of m, n, and p independently represents an integer of 0 or greater.

The molecular weight of the compound represented by the formula (A) is preferably between 50 and 500.

Preferably, in the formula (A), each of L₁, L₂, and L₃ independently represents a divalent linking group represented by —(CH₂)_{q}— wherein q represents an integer between 1 and 10.

Preferably, in the formula (A), m and p are 0, and n is 1.

In another aspect, the present invention provides a biosensor having a substrate, to the surface of which a linker having a structure represented by the following formula (B) is bound: 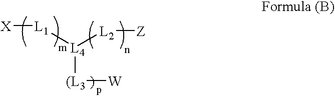
wherein X represents a group capable of binding to the surface of a biosensor or a functional group existing on the surface of the biosensor; W represents a group capable of binding to a physiologically active substance or a compound binding to the physiologically active substance; Z represents a group having an electric charge; each of L₁, L₂, and L₃ independently represents a divalent linking group; L₄ represents a trivalent or polyvalent group; each of m and n independently represents an integer of 0 or greater; and p represents a natural number.

The molecular weight of the linker represented by the formula (B) is preferably between 50 and 10,000.

Preferably, in the formula (B), each of L₁, L₂, and L₃ independently represents a divalent linking group represented by —(CH₂)_{q}— wherein q represents an integer between 1 and 10.

Preferably, in the formula (B), m is 0, p is 1, and n is 1.

Preferably, the substrate is a substrate of a metal surface or metal film.

Preferably, the metal surface or metal film consists of a free electron metal selected from the group consisting of gold, silver, copper, platinum, and aluminum.

Preferably, the substrate is a metal surface or metal film coated with a hydrophobic polymer or polyhydroxy polymer, or a metal surface or metal film having a self assembled membrane.

Preferably, the coating thickness of the hydrophobic polymer is between 0.1 nm and 500 nm.

Preferably, the biosensor of the present invention is used in non-electrochemical detection, and more preferably in surface plasmon resonance analysis.

In further another aspect, the present invention provides a method for producing the above-described biosensor of the present invention, which comprises a step of allowing a compound represented by the following formula (A) to react with a substrate, so as to bind them: 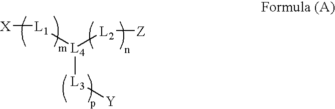
wherein X represents a group capable of binding to the surface of a biosensor or a functional group existing on the surface of the biosensor; Y represents a group capable of binding to a physiologically active substance or a compound binding to the physiologically active substance; Z represents a group having an electric charge; each of L₁, L₂, and L₃ independently represents a divalent linking group; L₄ represents a trivalent group; and each of m, n, and p independently represents an integer of 0 or greater.

In further another aspect, the present invention provides a method for producing the above-described biosensor of the present invention, which comprises steps of allowing a compound represented by the following formula (B′) to react with a substrate, so as to bind them: and then allowing a compound represented by X₂-(L₅)q-W₁ to react therewith so as to bind them. 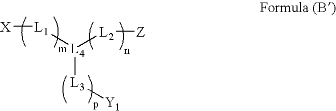
wherein X, Z, L₁, L₂, L₃, L₄, m, n, and p are as defined above; Y₁ represents a group capable of binding to a group represented by X₂; X₂ represents a group capable of binding to a group represented by Y₁; L₅ represents a divalent linking group; q represents a natural number, and W₁ represents a group capable of binding to a physiologically active substance or a compound binding to the physiologically active substance.

In further another aspect, the present invention provides the biosensor according to the present invention, wherein a physiologically active substance is bound to the surface by covalent bonding.

In further another aspect, the present invention provides a method for immobilizing a physiologically active substance on a biosensor, which comprises a step of allowing a physiologically active substance to come into contact with the biosensor according to the present invention, so as to allow said physiologically active substance to bind to the surface of said biosensor via a covalent bond.

In further another aspect, the present invention provides a method for detecting or measuring a substance interacting with a physiologically active substance, which comprises a step of allowing a test substance to come into contact with the biosensor according to the present invention to the surface of which the physiologically active substance binds via a covalent bond.

Preferably, the substance interacting with the physiologically active substance is detected or measured by a non-electrochemical method. Particularly preferably, the substance interacting with the physiologically active substance is detected or measured by surface plasmon resonance analysis.

### BEST MODE FOR CARRYING OUT THE INVENTION

The embodiments of the present invention will be described below.

The biosensor of the present invention is characterized in that it has a substrate, to the surface of which a compound represented by the following formula (A) is bound: 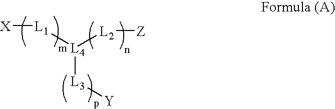
wherein X represents a group capable of binding to the surface of a biosensor or a functional group existing on the surface of the biosensor; Y represents a group capable of binding to a physiologically active substance or a compound binding to the physiologically active substance; Z represents a group having an electric charge; each of L₁, L₂, and L₃ independently represents a divalent linking group; L₄ represents a trivalent group; and each of m, n, and p independently represents an integer of 0 or greater.

In another aspect, the biosensor of the present invention is characterized in that it has a substrate, to the surface of which a linker having a structure represented by the following formula (B) is bound: 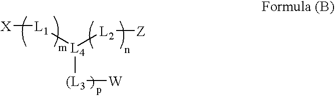
wherein X represents a group capable of binding to the surface of a biosensor or a functional group existing on the surface of the biosensor; W represents a group capable of binding to a physiologically active substance or a compound binding to the physiologically active substance; Z represents a group having an electric charge; each of L₁, L₂, and L₃ independently represents a divalent linking group; L₄ represents a trivalent or polyvalent group; each of m and n independently represents an integer of 0 or greater; and p represents a natural number.

In the formulas (A) and (B), X represents a group capable of binding to the surface of a biosensor or a functional group existing on the surface of the biosensor. When the biosensor surface is a metal, examples of X may include a sulfur atom and an amino group. Preferred examples of a group capable of reacting with a functional group existing on the biosensor surface may include a halogen atom, an amino group, an amino group protected by a protecting group, a carboxyl group, a carbonyl group having a leaving group, a hydroxyl group, a hydroxyl group protected by a protecting group, an aldehyde group, —NHNH₂, —N═C═O, —N═C═S, an epoxy group, and a vinyl group. X is particularly preferably an amino group or an amino group protected by a protecting group.

The term “protecting group” is used herein to mean a group capable of deprotecting a target group in a reaction system, so as to allow it to form a functional group. Examples of a protecting group for an amino group may include a tert-butyloxycarbonyl group (Boc), a 9-fluorenyl-methyloxycarbonyl group (Fmoc), a nitrophenylsulphenyl group (Nps), and a dithiasuccinyl group (Dts). In addition, an acyl group may be an example of a protecting group for a hydroxyl group. Examples of a leaving group used herein may include a halogen atom, an alkoxy group, an aryloxy group, an alkylcarbonyloxy group, an arylcarbonyloxy group, a halogenated alkylcarbonyloxy group, an alkylsulfonyloxy group, a halogenated alkylsulfonyloxy group, and an arylsulfonyloxy group. Moreover, an ester group generated as a result of the combination of carboxylic acid, a known dehydrating condensing reagent (e.g. carbodiimide), and an N-hydroxy compound, is also preferably used as a leaving group.

Y in the formula (A) and W in the formula (B) each represent a group capable of immobilizing a physiologically active substance or capable of binding to a compound binding to the physiologically active substance. Y and W are preferably a halogen atom, an amino group, an amino group protected by a protecting group, a carboxyl group, a carbonyl group having a leaving group, a hydroxyl group, a hydroxyl group protected by a protecting group, an aldehyde group, —NHNH₂, —N═C═O, —N═C═S, an epoxy group, or a vinyl group. As a protecting group and a leaving group, the same above groups may be used. Y and W are particularly preferably a carboxyl group.

In the formulas (A) and (B), Z represents a group having an electric charge. In this case, Z may form salts having counterions. Examples of a group having an electronegative charge may be carboxylate, sulfonate, sulfinate, and phosphate. Examples of a group having an electropositive charge preferably used herein may include an onium salt (ammonium salt, pyridium salt, sulfonium salt, phosphonium salt, etc.), a substituted or unsubstituted amino group, and a guanidyl group. When Z is a group having an electronegative charge, a sulfonic acid group (—SO₃) (or sulfonate) is particularly preferable. When Z is a group having an electropositive charge, an onium salt is particularly preferable.

In the formulas (A) and (B), each of L₁, L₂, and L₃ independently represents a divalent linking group.

Each of L₁, L₂, and L₃ may be either linear, or branched at some midpoint. Each of L₁, L₂, and L₃ is preferably a linking group having a main chain containing 1 to 10 carbon atoms, and more preferably a divalent linking group represented by —(CH₂)_{q}— wherein q represents an integer between 1 and 10.

In the formulas (A) and (B), L₄ represents a trivalent or polyvalent group (“polyvalent” means that the number of valence is more than 3). It is preferably a carbon atom (that is, a trivalent group represented by CH), an aromatic ring (e.g. a benzene ring, a naphthalene ring, etc.), or a heterocyclic ring.

In the formula (A), each of m, n, and p independently represents an integer of 0 or greater. It preferably represents an integer between 0 and 5, more preferably represents an integer between 0 and 3, and particularly preferably represents 0 or 1.

In the formula (B), each of m and n independently represents an integer of 0 or greater. It preferably represents an integer between 0 and 5, more preferably represents an integer between 0 and 3, and particularly preferably represents 0 or 1. In the formula (B), p represents a natural number, preferably a natural number between 1 and 5, further preferably a natural number between 1 and 3, particularly preferably 1.

The molecular weight of the compound represented by the formula (A) is preferably between 50 and 500, more preferably between 50 and 300, and particularly preferably between 100 and 200.

The molecular weight of the compound represented by the formula (B) is preferably between 50 and 10000, more preferably between 50 and 5000, and particularly preferably between 100 and 1000.

In the formula (B′), X₂ represents a group capable of binding to a group represented by Y₁, and examples thereof include a halogen atom, an amino group, an amino group protected by a protecting group, a carboxyl group, a carbonyl group having a leaving group, a hydroxyl group, a hydroxyl group protected by a protecting group, an aldehyde group, —NHNH₂, —N═C═O, —N═C═S, an epoxy group, or a vinyl group. As a protecting group and a leaving group, the same above groups may be used.

In the present invention, L₅ represents a divalent linking group. In -(L₅)q-, the number of atom of a main chain is preferably 5 or more, more preferably 5 to 800. L₅ is represented by, for example, —(CH₂)ₜ— wherein t represents an integer between 5 and 800.

q represents an integer of 1 or more, preferably an integer of 1 to 5, more preferably an integer of 1 to 3, and particularly preferably 1.

W1 has the same definition as that of the aforementioned W.

In the present invention, a compound having a group (an electric charge) represented by Z is introduced onto a substrate, and then a linker may be further introduced. Alternatively, a compound having an electric charge is reacted with a linker outside the system, and them the reaction product may be introduced onto a substrate.

Specific examples of a compound represented by the formula (A) may include the following compounds.

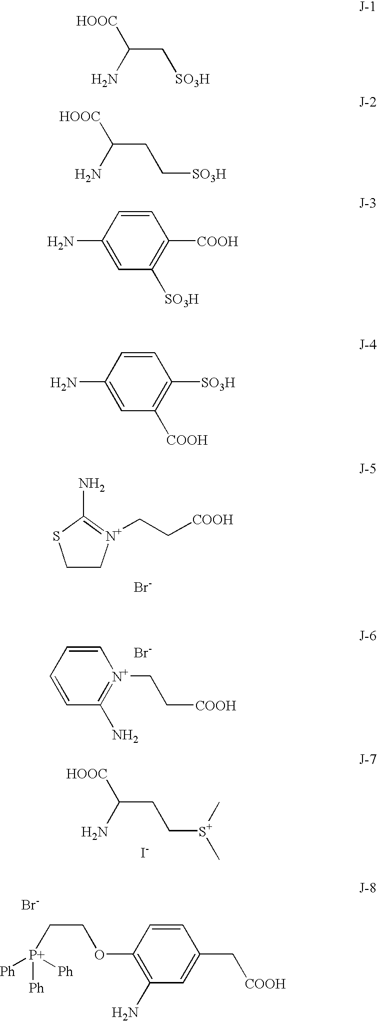

Specific examples of a linker having a structure represented by the formula (B) may include the following compounds.

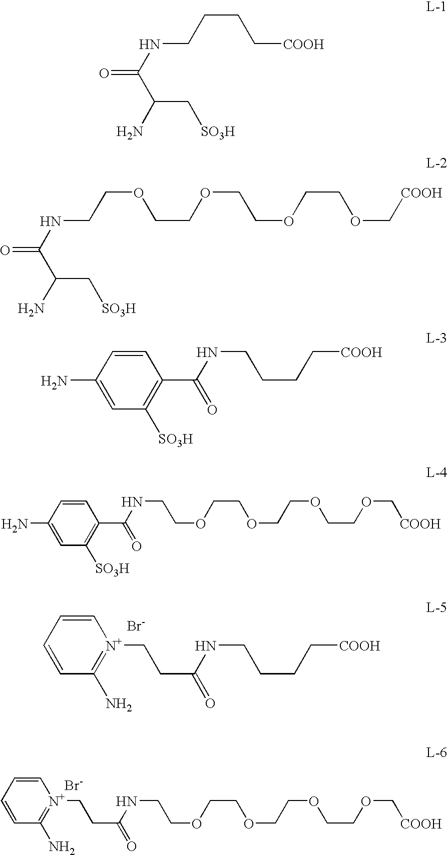

The biosensor of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

In the biosensor of the present invention, a metal surface or a metal film can be used as a substrate. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 0.1 nm and 500 nm, and particularly preferably between 1 nm and 200 nm. If the thickness exceeds 500 nm, the surface plasmon phenomenon of a medium cannot be sufficiently detected. Moreover, when an interstitial layer consisting of chrome or the like is provided, the thickness of the interstitial layer is preferably between 0.1 nm and 10 nm.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and having excellent workability are preferably used.

Preferably in the present invention, the substrate is a metal surface or metal film coated with a hydrophobic polymer or polyhydroxy polymer, or a metal surface or metal film having a self assembled membrane. When a linker having a structure represented by the formula (B) is used, the substrate is preferably a metal surface or metal film coated with a hydrophobic polymer. The hydrophobic polymer, polyhydroxy polymer, and self assembled membrane are mentioned below.

The hydrophobic polymer used in the present invention is a polymer having no water-absorbing properties. Its solubility in water (at 25° C.) is 10% or less, more preferably 1% or less, and most preferably 0.1% or less.

A hydrophobic monomer which forms a hydrophobic polymer can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Examples of a hydrophobic polymer that is preferably used in the present invention may include polystyrene, polyethylene, polypropylene, polyethylene terephthalate, polyvinyl chloride, polymethyl methacrylate, polyester, and nylon.

A substrate is coated with a hydrophobic polymer according to common methods. Examples of such a coating method may include spin coating, air knife coating, bar coating, blade coating, slide coating, curtain coating, spray method, evaporation method, cast method, and dip method.

In the dip method, coating is carried out by contacting a substrate with a solution of a hydrophobic polymer, and then with a liquid which does not contain the hydrophobic polymer. Preferably, the solvent of the solution of a hydrophobic polymer is the same as that of the liquid which does not contain said hydrophobic polymer.

In the dip method, a layer of a hydrophobic polymer having an uniform coating thickness can be obtained on a surface of a substrate regardless of inequalities, curvature and shape of the substrate by suitably selecting a coating solvent for hydrophobic polymer.

The type of coating solvent used in the dip method is not particularly limited, and any solvent can be used so long as it can dissolve a part of a hydrophobic polymer. Examples thereof include formamide solvents such as N,N-dimethylformamide, nitrile solvents such as acetonitrile, alcohol solvents such as phenoxyethanol, ketone solvents such as 2-butanone, and benzene solvents such as toluene, but are not limited thereto.

In the solution of a hydrophobic polymer which is contacted with a substrate, the hydrophobic polymer may be dissolved completely, or alternatively, the solution may be a suspension which contains undissolved component of the hydrophobic polymer. The temperature of the solution is not particularly limited, so long as the state of the solution allows a part of the hydrophobic polymer to be dissolved. The temperature is preferably −20° C. to 100° C. The temperature of the solution may be changed during the period when the substrate is contacted with a solution of a hydrophobic polymer. The concentration of the hydrophobic polymer in the solution is not particularly limited, and is preferably 0.01% to 30%, and more preferably 0.1% to 10%.

The period for contacting the solid substrate with a solution of a hydrophobic polymer is not particularly limited, and is preferably 1 second to 24hours, and more preferably 3 seconds to 1 hour.

As the liquid which does not contain the hydrophobic polymer, it is preferred that the difference between the SP value (unit: (J/cm³)^{1/2}) of the solvent itself and the SP value of the hydrophobic polymer is 1 to 20, and more preferably 3 to 15. The SP value is represented by a square root of intermolecular cohesive energy density, and is referred to as solubility parameter. In the present invention, the SP value 6 was calculated by the following formula. As the cohesive energy (Ecoh) of each functional group and the mol volume (V), those defined by Fedors were used (R.F.Fedors,Polym.Eng.Sci., 14(2)NP147, P472(1974)).
Δ=(ΣEcoh/ΣV)^{1/2}

Examples of the SP values of the hydrophobic polymers and the solvents are shown below;
Solvent: 2-phenoxyethanol: 25.3 against polymethylmethacrylate-polystyrene copolymer (1:1):21.0
Solvent: acetonitrile:22.9 against polymethylmethacrylate:20.3
Solvent: toluene:18.7 against polystyrene:21.6

The period for contacting a substrate with a liquid which does not contain the hydrophobic polymer is not particularly limited, and is preferably 1 second to 24 hours, and more preferably 3 seconds to 1 hour. The temperature of the liquid is not particularly limited, so long as the solvent is in a liquid state, and is preferably −20° C. to 100° C. The temperature of the liquid may be changed during the period when the substrate is contacted with the solvent. When a less volatile solvent is used, the less volatile solvent may be substituted with a volatile solvent which can be dissolved in each other after the substrate is contacted with the less volatile solvent, for the purpose of removing the less volatile solvent.

The coating thickness of a hydrophobic polymer is not particularly limited, but it is preferably between 0.1 nm and 500 nm, and particularly preferably between 1 nm and 300 nm.

Examples of a polyhydroxy polymer used in the present invention may include polysaccharides (e.g. agarose gel, dextran, carrageenan, alginic acid, starch, and cellulose), and synthetic polymers (e.g. polyvinyl alcohol). In the present invention, polysaccharides are preferably used, and dextran is most preferable.

In the present invention, a polyhydroxy polymer having a mean molecular weight between 10,000 and 2,000,000 is preferably used. A polyhydroxy polymer having a mean molecular weight preferably between 20,000 and 2,000,000, more preferably between 30,000 and 1,000,000, and most preferably between 200,000 and 800,000, can be used.

For example, a polyhydroxy polymer is allowed to react with bromoacetic acid under basic conditions, so that it can be carboxylated. By controlling reaction conditions, a certain ratio of hydroxy groups contained in a polyhydroxy compound at an initial stage can be carboxylated. In the present invention, 1% to 90% hydroxy groups can be carboxylated, for example. The carboxylation rate of a surface coated with any given polyhydroxy polymer can be calculated by the following method. Using a di-tert-butylcarbodiimide/pyridine catalyst, the surface of a film is subjected to gas phase modification with trifluoroethanol at 50° C. for 16 hours. Thereafter, the amount of fluorine derived from trifluoroethanol is measured by ESCA (electron spectroscopy for chemical analysis), and the ratio between the amount of fluorine and the amount of oxygen on the film surface (hereinafter referred to as F/O value) is calculated. A theoretical F/O value obtained when all hydroxy groups have been carboxylated is set at 100%. Then, a F/O value obtained by carboxylation under certain conditions is measured. Thus, a carboxylation rate at that time can be calculated.

A polyhydroxy polymer can be attached to a metal film via an organic molecule X¹-R¹-Y¹. Such an organic molecule X¹-R¹-Y¹ will be described in detail.

X¹ is a group having ability to bind to a metal film. Specifically, asymmetrical or symmetrical sulfide (—SSR¹¹Y¹¹, —SSR¹Y¹), sulfide (—SR¹¹Y¹¹, —SR¹Y¹), diselenide (—SeSeR¹¹Y¹¹, —SeSeR¹Y¹), selenide (—SeR¹¹Y¹¹, —SeR¹Y¹), thiol (—SH), nitrile (—CN), isonitrile, nitro (—NO₂), selenol (—SeH), a trivalent phosphorus compound, isothiocyanate, xanthate, thiocarbamate, phosphine, thioacid, and dithioacid (—COSH, —CSSH) are preferably used.

R¹ (and R¹¹) are discontinued by hetero atoms in some cases. For a moderately dense load, these are preferably straight chains (that are not branched), and these are hydrocarbon chains containing double and/or triple bonds in some cases. Such a chain preferably has a length consisting of more than 10 atoms. A carbon chain may be perfluorinated in some cases.

Y¹ and Y¹¹ are groups for allowing a polyhydroxy polymer to bind with a metal film. Y′ and Y″ are preferably identical and have properties of capable of binding to a polyhydroxy polymer directly or after activation. Specifically, a hydroxyl, carboxyl, amino, aldehyde, hydrazide, carbonyl, epoxy, or vinyl group can be used.

Specific examples of an organic molecule X¹-R¹-Y¹ used herein may include 10-carboxy-1-decanethiol, 4,4′-dithiodibutyric acid, 11-hydroxy-1-undecanethiol, and 11-amino-1-undecanethiol.

A sulfur compound such as thiol or disulfide spontaneously adsorbs on a precious metal substrate such as gold, so as to provide an ultra-thin membrane with a size of a single molecule. In addition, since an aggregate thereof has a sequence that depends on the crystal lattice of a substrate or the molecular structure of an admolecule, it is called a self assembled membrane. In the present invention, 7-carboxy-1-heptanethiol, 10-carboxy-1-decanethiol, 4,4′-dithiodibutyric acid, 11-hydroxy-1-undecanethiol, 11-amino-1-undecanethiol, or the like can be used as such a self assembled membrane.

A substrate used in the present invention may further have a linker for allowing the compound represented by the formula (A) to bind to the substrate.

A specific example of a linker used in the present invention is a compound represented by the following formula (4):
X²⁰-L²⁰-Y²⁰ (4)
wherein X²⁰ represents a group capable of reacting with a hydrophobic polymer, a polyhydroxy polymer, or a functional group contained in a self assembled membrane; L²⁰ represents a divalent linking group; and Y²⁰ represents a group capable of reacting with a compound represented by the formula (A), so as to form a covalent bond.

In formula (4), X²⁰ represents a group capable of reacting with a hydrophobic polymer, a polyhydroxy polymer, or a functional group contained in a self assembled membrane. Preferred examples may include a halogen atom, an amino group, an amino group protected by a protecting group, a carboxyl group, a carbonyl group having a leaving group, a hydroxyl group, a hydroxyl group protected by a protecting group, an aldehyde group, —NHNH₂, —N═C═O, —N═C═S, an epoxy group, and a vinyl group.

The term “protecting group” is used herein to mean a group capable of deprotecting a target group in a reaction system, so as to allow it to form a functional group. Examples of a protecting group for an amino group may include a tert-butyloxycarbonyl group (Boc), a 9-fluorenyl-methyloxycarbonyl group (Fmoc), a nitrophenylsulphenyl group (Nps), and a dithiasuccinyl group (Dts).

In addition, an acyl group may be an example of a protecting group for a hydroxyl group.

Examples of a leaving group used herein may include a halogen atom, an alkoxy group, an aryloxy group, an alkylcarbonyloxy group, an arylcarbonyloxy group, a halogenated alkylcarbonyloxy group, an alkylsulfonyloxy group, a halogenated alkylsulfonyloxy group, and an arylsulfonyloxy group.

Moreover, an ester group generated as a result of the combination of carboxylic acid, a known dehydrating condensing reagent (e.g. carbodiimide), and an N-hydroxy compound, is also preferably used as a leaving group.

In formula (4), L²⁰ represents a divalent linking group. The total number of atoms contained in L is preferably between 2 and 1,000. Preferred examples of L²⁰ may include a substituted or unsubstituted alkyl group, a substituted or unsubstituted alkyleneoxy group, a substituted or unsubstituted aryleneoxy group, and a divalent linking group formed by binding X²⁰ in formula (4) to another molecule Y²⁰ and continuously repeating such a structure.

In formula (4), Y²⁰ represents a group capable of reacting with the compound represented by the formula (A), so as to form a covalent bond. Preferred examples of Y²⁰ may include a halogen atom, an amino group, an amino group protected by a protecting group, a carboxyl group, a carbonyl group having a leaving group, a hydroxyl group, a hydroxyl group protected by a protecting group, an aldehyde group, —NHNH₂, —N═C═O, —N═C═S, an epoxy group, and a vinyl group.

As a protecting group and a leaving group, the same above groups may be used.

Specific examples of the compound represented by formula (4) will be given below. However, compounds represented by formula (4) used in the present invention are not limited to such examples.

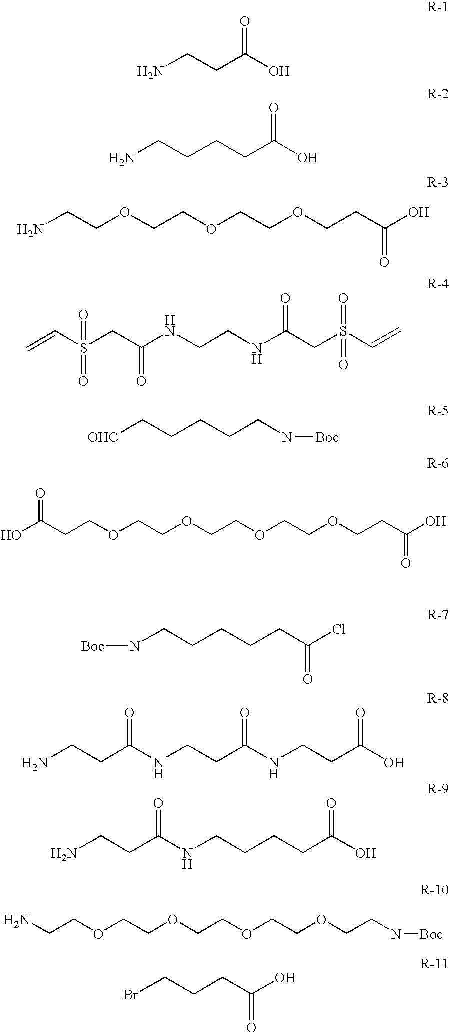

It is preferable that the biosensor of the present invention has a functional group capable of immobilizing a physiologically active substance on the outermost surface of the substrate. The term “the outermost surface of the substrate” is used herein to mean “the surface, which is farthest from the substrate,” and more specifically, it means “the surface, which is farthest from the surface in a hydrophobic polymer applied on a substrate.”

The thus obtained substrate for sensor can immobilize a physiologically active substance on a metal surface or metal film by covalently binding a physiologically active substance via a functional group Y in the aforementioned formula (A).

A physiologically active substance immobilized on the surface for the biosensor of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against O antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

A biosensor to which a physiologically active substance is immobilized as described above can be used to detect and/or measure a substance which interacts with the physiologically active substance.

In the present invention, it is preferable to detect and/or measure an interaction between a physiologically active substance immobilized on the surface used for a biosensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (θSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (θSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (θSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (θSP) is measured periodically when a certain period of time has elapsed.

If the test substance contained in the sample solution is bound to the sensing substance, the refractive index of the sensing substance is changed by this binding over time. Accordingly, the above attenuated total reflection angle (θSP) is measured periodically after the elapse of a certain time, and it is determined whether or not a change has occurred in the above attenuated total reflection angle (θSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (θSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (θSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (θSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

When the biosensor of the present invention is used in surface plasmon resonance analysis, it can be applied as a part of various surface plasmon measurement devices described above.

The present invention will be further specifically described in the following examples. However, the examples are not intended to limit the scope of the present invention.

### EXAMPLES

### Example A-1

### Production of Chip Used for Biosensors

(1) Production of Chip Used for Biosensors Coated With Polymethyl Methacrylate

A cover glass with a size of 1 cm×1 cm, which had been coated with gold via evaporation resulting in a gold film with a thickness of 50 nm, was treated with a Model-208 UV-ozone cleaning system (TECHNOVISION INC.) for 30 minutes. Thereafter, it was placed in a spin coater (MODEL ASS-303; manufactured by ABLE) and then rotated at 1,000 rpm. 50 μl of a methyl ethyl ketone solution containing polymethyl methacrylate (2 mg/ml) was added dropwise to the center of the cover glass coated with gold via evaporation, and 2 minutes later, the rotation was terminated. The thickness of the film was measured by the ellipsometry method (In-Situ Ellipsometer MAUS-101; manufactured by Five Lab). As a result, the thickness of the polymethyl methacrylate film was found to be 20 nm. This sample is called a PMMA surface chip.

(2) Introduction of COOH Group Into PMMA Surface

The cover glass coated with polymethyl methacrylate as produced above was immersed in an NaOH aqueous solution (1 N) at 40° C. for 16 hours, followed by washing with water 3 times. This sample is called a PMMA/COOH surface chip.

(3) Production of Surface Having Linker

The PMMA/COOH surface chip as produced above was immersed in 2 ml of a mixed solution of 1-ethyl-2,3-dimethylaminopropylcarbodiimide (400 mM) and N-hydroxysuccinimide (100 mM) for 60 minutes. Thereafter, it was immersed in 2 ml of 5-aminovaleric acid solution (1 M, adjusted to be pH 8.5) for 16 hours. The resultant product was finally washed with water 5 times. This sample is called a PMMA/Val surface chip.

(4) Production of Chip Used for Biosensors Coated With SAM Compound (7-carboxy-1-heptanethiol) (SAM: Self Assembled Membrane) A cover glass with a size of 1 cm×1 cm that had been coated with gold of 50 nm via evaporation was treated with an ozone cleaning system for 30 minutes. Thereafter, it was immersed in an ethanol solution containing 1 mM 7-carboxy-1-heptanethiol (Dojindo Laboratories), so as to conduct a surface treatment at 25° C. for 18 hours. Thereafter, the resultant product was washed at 40° C. with ethanol 5 times, with an ethanol/water mixed solution 1 time, and then with water 5 times. The thus obtained sample is called an SAM surface chip.

(5) Production of Surface On Which Physiologically Active Substance is Immobilized

The below-mentioned reaction was carried out on each of the PMMA/Val surface chip and the SAM surface chip, so as to produce the surface of the present invention on which a physiologically active substance is immobilized. Samples treated as follows are called a PMMA/Val/Cys-S surface chip and a SAM/Cys-S surface chip, respectively.

Namely, each of the PMMA/Val surface chip and the SAM surface chip was immersed in 2 ml of a mixed solution of 1-ethyl-2,3-dimethylaminopropylcarbodiimide (400 mM) and N-hydroxysuccinimide (100 mM) for 60 minutes, and it was then immersed in 2 ml of an L-cysteic acid (Wako Pure Chemical Industries, Ltd.) aqueous solution (1M, adjusted to be pH 8.5) for 16 hours. The resultant product was finally washed with water 5 times.

### Example A-2

### Evaluation of Performance of Chips Used for Biosensors

(1) Measurement of the Amount of Protein Immobilized

The amount of a physiologically active substance immobilized on the surface of a biosensor is preferably large to detect a substance binding to the physiologically active substance. The amount of Protein A (manufactured by Nacalai Tesque, Inc.) was measured using the following samples 1-1 to 1-4:
Sample 1-1: PMMA/Val surface chip (produced by the method described in Example A-1 (3))
Sample 1-2: SAM surface chip (produced by the method described in Example A-1 (4))
Sample 1-3: PMMA/Val/Cys-S surface chip (produced by the method described in Example A-1 (5))
   Sample 1-4: SAM/Cys-S surface chip (produced by the method described in Example A-1 (5))

The chip of each of the aforementioned samples 1-1 to 1-4 was placed on the cartridge block of a commercially available surface plasmon resonance biosensor (BIACORE 3000; manufactured by Biacore). Thereafter, 150 μl of a mixed solution of 1-ethyl-2,3-dimethylaminopropylcarbodiimide (400 mM) and N-hydroxysuccinimide (100 mM) was fed to a measurement cell at a flow rate of 10 μl/min. Thereafter, 150 μl of a solution (an acetate buffer, pH 5.5) containing 0.1 mg/ml Protein A (manufactured by Nacalai Tesque, Inc.) was fed to the measurement cell at a flow rate of 10 μl/min. Thereafter, 150 μl of an ethanol amine/HCl solution (1M, pH 8.5) was fed to the measurement cell at a flow rate of 10 μl/min. Subsequently, in order to eliminate a Protein A portion that had not covalently bound, 10 μl of 10 mM NaOH aqueous solution was fed to the measurement cell twice at a flow rate of 10 μl/min, so as to wash it out. Before and after the injection of each solution, an HBS-EP buffer (manufactured by Biacore, pH 7.4) (0.01 mol/l HEPES (pH 7.4); 0.15 mol/l NaCl; 0.003 mol/l EDTA; and 0.005% by weight of Surfactant P20) was fed to the measurement cell at a flow rate of 10 μl/min.

The value obtained before the injection of the mixed solution of 1-ethyl-2,3-dimethylaminopropylcarbodiimide (400 mM) and N-hydroxysuccinimide (100 mM) was set at 0. The value obtained after washing with the NaOH solution was defined as the amount of Protein A immobilized (RU).

(2) Results

The measurement results of the amount of Protein A immobilized are shown in Table 1.

TABLE 1

| | Amount of Protein A | |
|---|---|---|
| | immobilized (RU) | Remarks |
| Sample 1-1 | 1,000 | Comparative example |
| Sample 1-2 | 800 | Comparative example |
| Sample 1-3 | 2,400 | Present invention |
| Sample 1-4 | 1,500 | Present invention |

From the results shown in Table 1, it is found that the structure of the present invention can provide a surface on which an extremely large amount of protein is immobilized.

### Example B-1

### Production of Chip Used for Biosensors

(1) Production of Chip Used for Biosensors Coated With Polymethyl Methacrylate

A cover glass with a size of 1 cm×1 cm, which had been coated with gold via evaporation resulting in a gold film with a thickness of 50 nm, was treated with a Model-208 UV-ozone cleaning system (TECHNOVISION INC.) for 30 minutes. Thereafter, it was placed in a spin coater (MODEL ASS-303; manufactured by ABLE) and then rotated at 1,000 rpm. 50 μl of a methyl ethyl ketone solution containing polymethyl methacrylate (2 mg/ml) was added dropwise to the center of the cover glass coated with gold via evaporation, and 2 minutes later, the rotation was terminated. The thickness of the film was measured by the ellipsometry method (In-Situ Ellipsometer MAUS-101; manufactured by Five Lab). As a result, the thickness of the polymethyl methacrylate film was found to be 20 nm. This sample is called a PMMA surface chip.

(2) Introduction of COOH Group Into PMMA Surface

The cover glass coated with polymethyl methacrylate as produced above was immersed in NaOH aqueous solution (1 N) at 40° C. for 16 hours, followed by washing with water 3 times. This sample is called a PMMA/COOH surface chip.

(3) Production of Surface Having Cysteic Acid (for the Present Invention)

The PMMA/COOH surface chip produced in (2) above was immersed in 2 ml of a mixed solution of 1-ethyl-2,3-dimethylaminopropylcarbodiimide (400 mM) and N-hydroxysuccinimide (100 mM) for 60 minutes. Thereafter, it was immersed in 2 ml of an L-cysteic acid (Wako Pure Chemical Industries, Ltd.) aqueous solution (1M, adjusted to be pH 8.5) for 16 hours. The resultant product was finally washed with water 5 times. This sample is called a PMMA/Cys-S surface chip.

(4) Production of Surface On Which a Physiologically Active Substance is Immobilized (for the present invention)

The PMMA/Cys-S surface chip produced in (3) above was immersed in 2 ml of a mixed solution of 1-ethyl-2,3-dimethylaminopropylcarbodiimide (400 mM) and N-hydroxysuccinimide (100 mM) for 60 minutes. Thereafter, it was immersed in 2 ml of a 5-aminovaleric acid solution (1M, adjusted to be pH 8.5) for 16 hours. The resultant product was finally washed with water 5 times. This sample is called a PMMA/Cys-S/Val surface chip.

### Example B-2

### Evaluation of Performance of Chips Used for Biosensors

(1) Measurement of the Amount of Protein Immobilized

The amount of a physiologically active substance immobilized on the surface of a biosensor is preferably large to detect a substance binding to the physiologically active substance. The amount of Protein A (manufactured by Nacalai Tesque, Inc.) was measured using the following sample 2-1:
Sample 2-1: PMMA/Cys-S/Val surface chip (produced by the method described in

### Example B-1(4))

The chip of the sample 2-1 was placed on the cartridge block of a commercially available surface plasmon resonance biosensor (BIACORE 3000; manufactured by Biacore). Thereafter, 150 μl of a mixed solution of 1-ethyl-2,3-dimethylaminopropylcarbodiimide (400 mM) and N-hydroxysuccinimide (100 mM) was fed to a measurement cell at a flow rate of 10 μl/min. Thereafter, 150 μl of a solution (an acetate buffer, pH 5.5) containing 0.1 mg/ml human serum albumin (manufactured by Sigma) was fed to the measurement cell at a flow rate of 10 μl/min. Thereafter, 150 μl of an ethanol amine/HCl solution (1M, pH 8.5) was fed to the measurement cell at a flow rate of 10 μl/min. Subsequently, in order to eliminate a Protein A portion that had not covalently bound, 10 μl of a 10 mM NaOH aqueous solution was fed to the measurement cell twice at a flow rate of 10 μl/min, so as to wash it out. Before and after the injection of each solution, an HBS-EP buffer (manufactured by Biacore, pH 7.4) (0.01 mol/l HEPES (pH 7.4); 0.15 mol/l NaCl; 0.003 mol/l EDTA; and 0.005% by weight of Surfactant P20) was fed to the measurement cell at a flow rate of 10 μl/min.

The value obtained before the injection of the mixed solution of 1-ethyl-2,3-dimethylaminopropylcarbodiimide (400 mM) and N-hydroxysuccinimide (100 mM) was set at 0. The value obtained after washing with the NaOH solution was defined as the amount of Protein A immobilized (RU).

(2) Results

The measurement result of the amount of human serum albumin immobilized is shown in Table 2.

TABLE 2

| | Amount of human serum | |
|---|---|---|
| | albumin immobilized (RU) | Remarks |
| Sample 2-1 | 2,200 | Present invention |

From the results shown in Table 2, it is found that the structure of the present invention can provide a surface on which a large amount of protein is immobilized.

### Effect of the Invention

Accordingly to the present invention, it becomes possible to provide a detection surface used for biosensors having an improved efficiency of immobilizing a physiologically substance.

1. A biosensor having a substrate, to the surface of which a compound represented by the following formula (A) is bound: 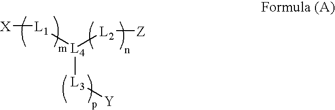
wherein X represents a group capable of binding to the surface of a biosensor or a functional group existing on the surface of the biosensor; Y represents a group capable of binding to a physiologically active substance or a compound binding to the physiologically active substance; Z represents a group having an electric charge; each of L₁, L₂, and L₃ independently represents a divalent linking group; L₄ represents a trivalent group; and each of m, n, and p independently represents an integer of 0 or greater.

2. The biosensor of claim 1 wherein the molecular weight of the compound represented by the formula (A) is between 50 and 500.

3. The biosensor of claim 1 wherein each of L₁, L₂, and L₃ independently represents a divalent linking group represented by —(CH₂)_{q}— wherein q represents an integer between 1 and 10.

4. The biosensor of claim 1 wherein m and p are 0, and n is 1.

5. A biosensor having a substrate, to the surface of which a linker having a structure represented by the following formula (B) is bound: 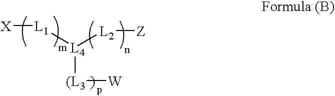
wherein X represents a group capable of binding to the surface of a biosensor or a functional group existing on the surface of the biosensor; W represents a group capable of binding to a physiologically active substance or a compound binding to the physiologically active substance; Z represents a group having an electric charge; each of L₁, L₂, and L₃ independently represents a divalent linking group; L₄ represents a trivalent or polyvalent group; each of m and n independently represents an integer of 0 or greater; and p represents a natural number.

6. The biosensor of claim 5 wherein the molecular weight of the linker represented by the formula (B) is preferably between 50 and 10,000.

7. The biosensor of claim 5 wherein each of L₁, L₂, and L₃ independently represents a divalent linking group represented by —(CH₂)_{q}— wherein q represents an integer between 1 and 10.

8. The biosensor of claim 5 wherein m is 0, p is 1, and n is 1.

9. The biosensor of claim 1 wherein the substrate is a substrate of a metal surface or metal film.

10. The biosensor of claim 9 wherein the metal surface or metal film consists of a free electron metal selected from the group consisting of gold, silver, copper, platinum, and aluminum.

11. The biosensor of claim 1 wherein the substrate is a metal surface or metal film coated with a hydrophobic polymer or polyhydroxy polymer, or a metal surface or metal film having a self assembled membrane.

12. The biosensor of claim 1 wherein the coating thickness of the hydrophobic polymer is between 0.1 nm and 500 nm.

13. The biosensor of claim 1 which is used in non-electrochemical detection.

14. The biosensor of claim 1 which is used in surface plasmon resonance analysis.

15. A method for producing the biosensor of claim 1, which comprises a step of allowing a compound represented by the following formula (A) to react with a substrate, so as to bind them: 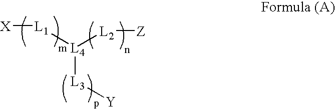
wherein X represents a group capable of binding to the surface of a biosensor or a functional group existing on the surface of the biosensor; Y represents a group capable of binding to a physiologically active substance or a compound binding to the physiologically active substance; Z represents a group having an electric charge; each of L₁, L₂, and L₃ independently represents a divalent linking group; L₄ represents a trivalent group; and each of m, n, and p independently represents an integer of 0 or greater.

16. A method for producing the biosensor of claim 5, which comprises steps of allowing a compound represented by the following formula (B′) to react with a substrate, so as to bind them: and then allowing a compound represented by X₂-(L₅)_{q}-W₁ to react therewith so as to bind them. 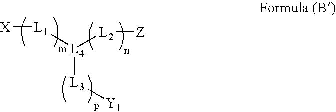
wherein X, Z, L₁, L₂, L₃, L₄, m, n, and p are as defined above; Y₁ represents a group capable of binding to a group represented by X₂; X₂ represents a group capable of binding to a group represented by Y₁; L₅ represents a divalent linking group; q represents a natural number, and W₁ represents a group capable of binding to a physiologically active substance or a compound binding to the physiologically active substance.

17. The biosensor of claim 1 wherein a physiologically active substance is bound to the surface by covalent bonding.

18. A method for immobilizing a physiologically active substance on a biosensor, which comprises a step of allowing a physiologically active substance to come into contact with the biosensor according to the present invention, so as to allow said physiologically active substance to bind to the surface of said biosensor via a covalent bond.

19. A method for detecting or measuring a substance interacting with a physiologically active substance, which comprises a step of allowing a test substance to come into contact with the biosensor of claim 1 to the surface of which the physiologically active substance binds via a covalent bond.

20. The method of claim 19 wherein the substance interacting with the physiologically active substance is detected or measured by a non-electrochemical method.

21. The method of claim 19 wherein the substance interacting with the physiologically active substance is detected or measured by surface plasmon resonance analysis.
